(19) United States
(12) Patent Application Publication
(10) Pub. No.: US 2024/0090830 A1
(43) Pub. Date: Mar. 21, 2024

(54) EEG-BASED METHODS FOR MEASURING PSYCHOACTIVE EFFECTS

(71) Applicant: Zentrela Inc., Hamilton, Ontario (CA)

(72) Inventors: Israel Gasperin Haaz, Hamilton, Ontario (CA); Weikai Qi, North York, Ontario (CA); Daniel Joseph Bosnyak, Hamilton, Ontario (CA)

(73) Assignee: Zentrela Inc., Hamilton, Ontario (CA)

(21) Appl. No.: 17/768,705

(22) PCT Filed: Oct. 16, 2020
(86) PCT No.: PCT/CA2020/051390

§ 371 Date: Apr. 13, 2022

Related U.S. Application Data
(60) Provisional application No. 62/923,811, filed on Oct. 21, 2019.

(51) Int. Cl. A61B 5/00; A61B 5/372

(52) U.S. Cl. CPC A61B 5/4848; A61B 5/372; A61B 5/7225; A61B 5/7267; A61B 5/7271; A61B 2503/20

(57) ABSTRACT

One or more algorithms are used to, in respect of each of a plurality of EEG snippets from a subject, allocate to each snippet a class selected from one or more classes, each class being associated with drug impairment level. The one or more algorithms are each defined by a model and a set of parameters and have been previously trained with a plurality of labelled EEG snippets, the labelled snippets including, for each of the classes, at least one snippet associated with a test subject experiencing the level of drug impairment associated with said each class. A statistical model is created which provides desired levels of sensitivity and accuracy based upon the proportion of snippets from test subjects having the known level of drug effect that falls within the associated class. Drug effect is defined by applying the statistical model to the classes allocated to the snippets.

### FIELD

The present invention relates to the field of drug testing.

### BACKGROUND OF THE INVENTION

The legalization of medicinal and recreational cannabis is happening around the world.

However, a reliable and commercially viable THC impairment test remains elusive: current drug tests for THC involve measuring levels of THC in oral fluids, breath, urine or blood but this is not a reliable indicator of cannabis impairment detection since a cannabis user can have high levels of THC residues in saliva, urine, breath and blood even several days post cannabis impairment.

### SUMMARY OF THE INVENTION

Fainting one aspect of the invention is a method for use with a psychoactive drug, the method comprising the steps of:
using a plurality of unique algorithms to, in respect of each of a plurality of EEG snippets, allocate to each snippet a class selected from a pair of classes, only one of the pair of classes being associated with use of the drug, each snippet being of a predetermined duration, the algorithms each being defined by a model and a set of parameters and having been previously trained with a plurality of labelled EEG snippets of the same predetermined duration, the labelled snippets including at least one snippet associated with a subject experiencing the psychoactive effect of the drug and labelled as the one of the pair of classes and at least one snippet associated with the subject when under no influence of the ding and labelled as the other of the pair of classes;
in respect of each snippet of the plurality of EEG snippets, making a finding of psychoactive effect if the one class is allocated to said each snippet in a proportion of the total classifications of the snippet that meets or exceeds a predetermined threshold; and
calculating the psychoactive effect of the drug based upon the proportion of EEG snippets found to have psychoactive effect.

According to another aspect, the plurality of labelled EEG snippets can be obtained by segmenting a plurality of EEG recordings.

According to another aspect, wherein the plurality of labelled EEG snippets can be that which remains after an EEG recording has been segmented and segments determined to be unreliable or noisy through conventional filtering techniques have been removed.

According to another aspect, the plurality of algorithms can include algorithms based upon each of a plurality of models.

According to another aspect, in respect of each model of the plurality, a plurality of algorithms can be defined, each of such plurality having a unique set of parameters.

According to another aspect, the algorithms can be machine learning algorithms.

According to another aspect, the labelled snippets can include: snippets associated with a plurality of subjects; and, in respect of each of the subjects, a plurality of snippets associated with the subject when the subject is experiencing the psychoactive effective of the drug and a plurality of snippets associated with the subject when under no influence of the drug.

Forming another aspect of the invention is apparatus comprising:
apparatus for collecting EEG data from a test subject and segmenting same into segmented EEG test data; and
a computing facility adapted to carry out the method for use with a psychoactive drug, wherein the plurality of EEG snippets used in the method are defined by the segmented EEG test data.

Forming another aspect of the invention is apparatus comprising:
apparatus for collecting EEG data from a test subject, segmenting same into segmented EEG test data and removing therefrom, using conventional filtering techniques, segments determined to be one or more of unreliable and noisy, thereby producing filtered segments; and
a computing facility adapted to carry out the method for use with a psychoactive drug, wherein the plurality of EEG snippets used in the method are defined by the filtered segments.

Forming another aspect of the invention is a method for use in a workplace, the method comprising the steps of:
in respect of a plurality of performances of a task, each task being performed by a person, in circumstances such that the task is performed by persons under varying levels of drug influence, capturing EEG data of the persons during the performance of the task;
choosing one of the performances as the minimum level of competence permissible in the workplace based upon an assessment of the performance for competency; and
using the method for use with a psychoactive drug to determine the extent to which the performer of the chosen task was under the influence of the drug and the permissible extent of drug influence in the workplace for persons responsible for carrying out the task.

Forming yet another aspect of the invention is a method for labelling a dosage of a psychoactive drug. This method comprising the steps of:
administering a dose of the drug to a person;
collecting EEG data from the person;
using the method for use with a psychoactive drug to calculate the maximum psychoactive effect the person experienced following the dose; and
labelling the dose with the calculated maximum.

Forming yet another aspect of the invention is a method for defining the effect of a psychoactive drug to desired levels of sensitivity and accuracy, the method comprising the steps of:
using one or more algorithms to, in respect of each of a plurality of EEG snippets from a subject, allocate to each snippet a class selected from one or more class, each class being associated with a level of drug impairment,
the one or more algorithms each being defined by a model and a set of parameters and having been previously trained with a plurality of labelled EEG snippets, the labelled snippets including, for each of the classes, at least one snippet associated with a test subject experiencing the level of drug impairment associated with said each class,
creating a statistical model which provides the desired levels of sensitivity and accuracy based upon the proportion of snippets from test subjects having the known level of drug effect that falls within the associated class; and
defining the effect of the drug upon the subject by applying the statistical model to the classes allocated to the snippets of the subject.

According to another aspect, the snippet length can be about 5000 milliseconds.

Advantages, features and characteristics of the invention will become apparent upon review of the following detailed description with reference to the appended drawings, the latter being briefly described hereinafter.

### BRIEF DESCRIPTION OF THE DRAWINGS

FIG. 1 shows apparatus according to an embodiment of the invention in use;

FIG. 2 is a view of the structure of FIG. 1 from the opposite side;

FIG. 3 shows the drug effects results of five participants;

FIG. 4 shows the results of a single participant smoking 7 puffs from a pipe;

FIG. 5 shows the mean results of 6 participants after various times after intake of a THC beverage containing either 2.5 mg or 10 mg of THC;

FIG. 6 shows an example of the data resampling with cubic spline interpolation; and

FIG. 7 shows accuracy, sensitivity and specificity calculated utilizing various thresholds.

### DETAILED DESCRIPTION

### Method

A non-limiting example of the inventive method for use with a psychoactive drug involves a plurality of EEG snippets, a plurality of labelled EEG snippets and a plurality of unique algorithms.

The plurality of EEG snippets is that which remains after an EEG recording of a subject has been segmented into snippets each of a predetermined duration and segments determined to be unreliable or noisy through conventional filtering techniques have been removed.

The plurality of labelled EEG snippets is that which remains after each of plurality of labelled EEG recordings has been segmented and segments determined to be unreliable or noisy through conventional filtering techniques have been removed, the labelled EEG recordings including labelled recordings of a plurality of subjects and more particularly including, in respect of each of the plurality of subjects: (i) a plurality of recordings made when the subject is experiencing the psychoactive effective of the drug and labelled as such; and (ii) a plurality of recordings made when the subject is under no influence of the drug and labelled as such.

The plurality of algorithms is a plurality of machine learning algorithms trained using the plurality of labelled EEG snippets, includes algorithms based upon each of a plurality of models and includes, in respect of each model of the plurality, a plurality of algorithms, each of such plurality having a unique set of parameters.

The method comprises the steps of:
using the plurality of unique algorithms to, in respect of each of the plurality of EEG snippets, allocate to each snippet a class selected from a pair of classes, only one of the pair of classes being associated with use of the drug;
in respect of each snippet of the plurality of EEG snippets, making a finding of psychoactive effect if the one class is allocated to said each snippet in a proportion of the total classifications of the snippet that meets or exceeds a predetermined threshold; and
calculating the psychoactive effects of the drug based upon the proportion of EEG snippets found to have psychoactive effects.

### Calibration

Thousands of EEG recordings were taken from a variety of subjects. The EEG device used was an 8-channel OpenBCI Cyton System with a sampling rate of 250 Hz at a resolution of 24 bits. g.tec GAMMAgel conductive gel was utilized to reduce electrode impedance below a predetermined threshold. Wet electrodes were placed at locations: right frontal lobe, left frontal lobe, right temporal lobe, left temporal lobe, right occipital lobe, left occipital lobe, right parietal lobe and left parietal lobe. The EEG recording length varied in time between 60 and 600 seconds. The subjects included males ranging in age from 19-68 and females ranging in age from 19-67. All subjects were prior users of cannabis. Subjects self-reported use levels over a wide range ranging from daily doses of about 600 mg THC to occasional, low dosage use.

Recordings were labelled for “under no drug influence” only when the subject arrived at the session, self-reported no recent drug use and the behavior of such subject, in the assessment of the EEG technician, was aligned with such self-assessment.

For the purpose of psychoactive data collection, cannabis in various forms was made available to the subjects who self-administered doses, in their personal discretion, varying from about 10 to 100 mg THC. Recordings were labelled for the presence of psychoactive effect only if drug consumption of the subject had been observed, when the subject self-reported a perception of being “completely stoned” or “really high”, and asserted that they would not feel comfortable driving in their current state.

The labelled recordings were segmented into 10 second labelled snippets which were passed through a band pass filter and a 60 Hz notch filter and then were analyzed across a number of characteristics including maximum peak and RMS voltage, minimum peak and RMS voltage, and various spectral properties, in order to come to a decision as to whether that particular segment was unreliable or reliable. Segments found to be unreliable were removed from the set of labelled segments.

The remaining labelled segments were used to train five models as shown in Table 1, thereby producing five algorithms.

TABLE 1

| | | | | GRID SEARCH |
|---|---|---|---|---|
| ALGORITHM | MODEL | CLASSIFIER | FEATURES | TYPE |
| 1 | P26_Model_1 | SVM | List 1 [Table 2] | Gridtest |
| 2 | P26_Model_1 | SVM | List 2 [Table 3] | Griddense |
| 3 | P26_Model_1 | SVM | List 1 [Table 2] | Griddense |
| 4 | P26_Model_1 | SVM | List 3 [Table 4] | Gridtest |
| 5 | P26_Model_1 | SVM | List 1 [Table 2] | griddense |

TABLE 2
LIST 1

| | | |
|---|---|---|
| cpsd-ch7ch8-f11.50 | psd-ch6-f36.00 | psd-ch2-f21.50 |
| cpsd-ch2ch7-f11.50 | cpsd-ch1ch7-f11.50 | psd-ch2-f4.50 |
| cpsd-ch1ch4-f10.00 | cpsd-ch1ch2-f11.50 | psd-ch6-f40.00 |
| psd-ch4-f1.50 | coherence-ch1ch3-f10.00 | coherence-ch6ch8-f19.50 |
| psd-ch3-f4.50 | psd-ch2-f3.50 | coherence-ch3ch7-f3.50 |
| coherence-ch1ch8-f1.50 | coherence-ch6ch7-f11.00 | coherence-ch6ch8-f10.00 |
| psd-ch6-f5.00 | psd-ch6-f43.00 | psd-ch5-f27.00 |
| psd-ch2-f18.00 | psd-ch6-f12.50 | psd-ch8-f2.00 |
| psd-ch6-f6.00 | psd-ch2-f6.00 | coherence-ch2ch3-f4.00 |
| psd-ch5-f0.50 | psd-ch3-f6.00 | coherence-ch6ch8-f37.50 |
| psd-ch4-f1.00 | coherence-ch1ch3-f10.50 | coherence-ch2ch6-f3.50 |
| psd-ch7-f13.00 | psd-ch6-f4.00 | psd-ch3-f6.50 |
| coherence-ch2ch3-f10.50 | psd-ch6-f5.50 | psd-ch1-f4.00 |
| cpsd-ch2ch8-f11.50 | psd-ch2-f12.00 | psd-ch5-f27.50 |
| psd-ch6-f13.50 | coherence-ch6ch8-f28.00 | psd-ch6-f3.00 |
| psd-ch6-f39.50 | coherence-ch3ch6-f3.50 | psd-ch3-f11.50 |
| coherence-ch6ch8-f9.50 | psd-ch1-f11.50 | psd-ch3-f18.00 |
| psd-ch1-f10.00 | cpsd-ch1ch8-f11.50 | cpsd-ch6cb7-f13.00 |
| psd-ch6-f13.00 | psd-ch6-f45.50 | psd-ch6-f16.50 |
| psd-ch8-f11.50 | psd-ch7-f48.00 | psd-ch6-f42.00 |
| psd-ch2-f11.50 | coherence-ch6ch8-f38.00 | psd-ch2-f5.50 |
| psd-ch6-f4.50 | cpsd-ch2ch8-f47.50 | psd-ch6-f38.00 |
| coherence-ch2ch3-f11.00 | psd-ch4-f41.50 | coherence-ch6ch8-f19.00 |
| coherence-ch6ch8-f47.00 | psd-ch2-f17.00 | psd-ch8-f1.50 |
| cpsd-ch6ch8-f13.00 | coherence-ch3ch8-f10.00 | cpsd-ch7ch8-f11.00 |
| coherence-ch2ch3-f11.50 | psd-ch7-f11.00 | psd-ch2-f17.50 |
| psd-ch5-f28.50 | psd-ch2-f18.50 | psd-ch6-f37.50 |
| psd-ch3-f4.00 | cpsd-ch1ch8-f10.00 | coherence-ch3ch8-f10.50 |
| psd-ch7-f11.50 | psd-ch8-f11.00 | coherence-ch1ch8-f2.50 |
| coherence-ch6ch7-f11.50 | psd-ch2-f10.50 | psd-ch3-f3.00 |
| coherence-ch2ch3-f4.50 | psd-ch2-f13.00 | cpsd-ch6ch8-f17.50 |
| psd-ch2-f6.50 | psd-ch7-f4.00 | psd-ch3-f44.50 |
| cpsd-ch2ch4-f10.00 | psd-ch3-f13.00 | |
| coherence-ch1ch4-f10.00 | psd-ch2-f4.00 | |

TABLE 3
LIST 2

| | | |
|---|---|---|
| coherence-ch2ch3-f4.00 | psd-ch3-f14.00 | coherence-ch3ch6-f12.00 |
| cpsd-ch7ch8-f14.00 | psd-ch6-f44.00 | coherence-ch2ch3-f14.00 |
| coherence-ch2ch3-f12.00 | psd-ch5-f28.00 | psd-ch6-f32.00 |
| psd-ch3-f4.00 | cpsd-ch6ch7-f18.00 | psd-ch2-f18.00 |
| cpsd-ch2ch7-f20.00 | psd-ch2-f4.00 | psd-ch7-f14.00 |
| psd-ch6-f4.00 | psd-ch3-f6.00 | psd-ch4-f44.00 |
| psd-ch6-f14.00 | psd-ch2-f14.00 | coherence-ch3ch8-f2.00 |
| coherence-ch1ch8-f2.00 | coherence-ch3ch8-f10.00 | cpsd-ch4ch5-f14.00 |
| psd-ch2-f16.00 | cpsd-ch2ch7-f22.00 | cpsd-ch6ch8-f14.00 |
| cpsd-ch2ch7-f14.00 | psd-ch6-f40.00 | cpsd-ch6ch7-f14.00 |
| psd-ch6-f42.00 | psd-ch5-f26.00 | coherence-ch2ch3-f16.00 |
| psd-ch1-f10.00 | epsd-ch5ch6-f12.00 | psd-ch2-f6.00 |
| psd-ch2-f22.00 | psd-ch4-f2.00 | coherence-ch3ch6-f46.00 |
| cpsd-ch6ch8-f4.00 | coherence-ch1ch8-f34.00 | psd-ch6-f12.00 |
| psd-ch4-f42.00 | coherence-ch2ch6-f4.00 | psd-ch4-f40.00 |
| coherence-ch6ch8-f46.00 | psd-ch6-f6.00 | psd-ch6-f50.00 |
| psd-ch6-f38.00 | coherence-ch1ch3-f10.00 | |

TABLE 4
LIST 3

| | | |
|---|---|---|
| psd-ch6-f3.00f6.00 | psd-ch6-f42.00f45.00 | psd-ch4-f39.00f42.00 |
| coherence-ch2ch3-f3.00f6.00 | coherence-ch1ch8-f0.50f3.00 | psd-ch5-f30.00f33.00 |
| coherence-ch2ch3-f9.00f12.00 | psd-ch6-f39.00f42.00 | coherence-ch6ch8-f27.00f30.00 |
| psd-ch6-f12.00f15.00 | psd-ch2-f21.00f24.00 | coherence-ch1ch3-f9.00f12.00 |
| psd-ch3-f3.00f6.00 | coherence-ch3ch8-19.00f12.00 | cpsd-ch4ch5-f15.00f18.00 |
| cpsd-ch2ch7-f18.00f21.00 | psd-ch2-f3.00f6.00 | cpsd-ch2ch3-f24.00f27.00 |
| psd-ch5-f27.00f30.00 | psd-ch2-f24.00f27.00 | coherence-ch6ch8-f18.00f21.00 |
| psd-ch2-f15.00f18.00 | cpsd-ch6ch7-f15.000f8.00 | coherence-ch3ch8-f0.50f3.00 |
| cpsd-ch5ch6-f12.00f15.00 | psd-ch4-f42.00f45.00 | psd-ch6-f30.00f33.00 |
| cpsd-ch2ch7-f21.00f24.00 | psd-ch6-f36.00f39.00 | coherence-ch3ch6-f27.00f30.00 |

### Testing

EEG data was recorded from a female subject, aged 28, who self-reported her level of cannabis psychoactive effect as 7 out of 10 at the time of recording using the method described here (https://www.ncbi.nlm.nih.gov/pmc/articles/PMC3624068). The recording was segmented into 10 second snippets which were passed through a band pass filter and a 60Hz notch filter and snippets that contained noise or otherwise appeared unreliable were removed.

Thereafter, a class was allocated to each snippet by each algorithm, only one [Y] of the pair of classes [Y/N] being associated with use of the drug, the results thereof being shown in Table 5

TABLE 5

| | Snippet | | | | | | | | | | | | | | | | | | | |
|---|---|---|---|---|---|---|---|---|---|---|---|---|---|---|---|---|---|---|---|---|
| Algorithm | 1 | 2 | 3 | 4 | 5 | 6 | 7 | 8 | 9 | 10 | 11 | 12 | 13 | 14 | 15 | 16 | 17 | 18 | 19 | 20 |
| 1 | | | | y | | Y | y | | | Y | y | Y | | y | Y | | | y | Y | |
| 2 | | y | y | y | | | Y | y | | y | | | x | y | Y | y | Y | Y | | |
| 3 | Y | | | Y | | Y | Y | | | Y | Y | Y | | Y | Y | | | Y | Y | |
| 4 | y | | Y | y | Y | Y | Y | Y | y | Y | | | | | Y | Y | Y | Y | Y | |
| 5 | | | | y | Y | y | Y | | | y | Y | y | | Y | Y | | | Y | y | |
| Total | 2 | 1 | 2 | 4 | 2 | 4 | 5 | 2 | 1 | 5 | 3 | 3 | 1 | 4 | 5 | 2 | 2 | 5 | 4 | |

Thereafter, in respect of each snippet of the plurality of EEG snippets, a finding of psychoactive effect was made if the one class is allocated to said each snippet in a proportion of the total classifications of the snippet that meets or exceeds a predetermined threshold of 40% [2], as all shown in Table 6

TABLE 6

| | Snippet | | | | | | | | | | | | | | | | | | | |
|---|---|---|---|---|---|---|---|---|---|---|---|---|---|---|---|---|---|---|---|---|
| Algorithm | 1 | 2 | 3 | 4 | 5 | 6 | 7 | 8 | 9 | 10 | 11 | 12 | 13 | 14 | 15 | 16 | 17 | 18 | 19 | 20 |
| Total | 2 | 1 | 2 | 4 | 2 | 4 | 5 | 2 | 1 | 5 | 3 | 3 | 1 | 4 | 5 | 2 | 2 | 5 | 4 | |
| Finding | Y | | Y | Y | Y | Y | Y | Y | | Y | Y | Y | | Y | Y | Y | Y | Y | Y | |

Finally, the psychoactive effect of the drug was calculated at 80%, based upon the proportion of EEG snippets found to have psychoactive effect [16 “Y” out of 20]

### Apparatus

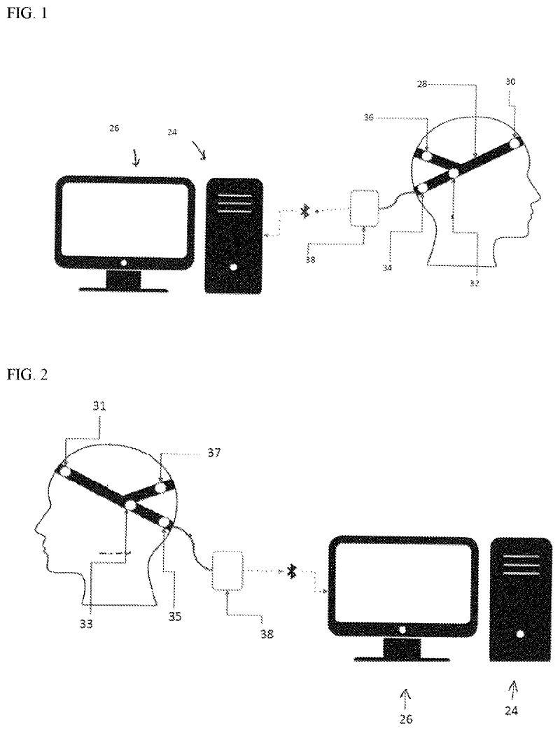

A non-limited example of apparatus 20 according to the invention is shown in FIG. 1 and FIG. 2 and will be understood to comprise EEG apparatus 22, a computing facility 24 and an output facility 26.

The EEG apparatus 22 is for collecting EEG data from a test subject, segmenting same into segmented EEG test data and removing therefrom, using conventional filtering techniques, segments determined to be one or more of unreliable and noisy, thereby producing filtered segments and in this embodiment includes a headset 28, a plurality of electrodes 30-37, a printed circuit board 38 with wireless functionality and a plurality of wires 40 coupling the electrodes to the board. The electrodes 30-37 are disposed, respectively, at the right front, left front, right temporal, left temporal, right occipital, left occipital, right parietal lobe and left parietal lobes.

The computing facility 24 in this embodiment is a CPU having wireless connectivity to the board and adapted to carry out the method, wherein the plurality of EEG snippets used in the method are defined by the filtered segments.

The output facility 26 is a screen coupled to the CPU and adapted to deliver to the user of the apparatus an indication of the psychoactive effect.

### Workplace Method

A non-limiting example of the method for use in a workplace comprises the steps of:
in respect of a plurality of performances of a task, each task being performed by a person, in circumstances such that the task is performed by persons under varying levels of drug influence, capturing EEG data of the persons during the performance of the task;
choosing one of the performances as the minimum level of competence permissible in the workplace based upon an assessment of the performance for competency; and
using the method to determine the extent to which the performer of the chosen task was under the influence of the drug and the permissible extent of drug influence in the workplace for persons responsible for carrying out the task.

Persons of ordinary skill will appreciate that the foregoing could be used, for example, by the workplace health and safety committee of an industrial employer. Working in consultation, employer and employee representatives could create a test, for example, for a forklift operator, and invite operators that use marijuana to operate the vehicles (in controlled and safe situations) to perform the test at various levels of impairment while EEG data was contemporaneously taken. The representatives could then sort the results into operators which appeared to have performed the task effectively and those that did not. Of those operators that had not performed the task effectively, the psychoactive effects could be calculated, and of those, the lowest effect could be selected as the cut-off point at which an employee could be prohibited from work.

### Labelling Method

A non-limiting example of the method for labelling the dosage of a psychoactive comprises the steps of:
administering a known quantity (‘a dose’) of the drug to a person;
collecting EEG data from the person;
using the inventive method to calculate the maximum psychoactive effect the person experienced following the dose; and
labelling the dose with the calculated maximum.

Persons of ordinary skill will readily appreciate that, by the consistent use of such method across a series of products, consumers that wish to use a drug can more safely self-administer, by comparing previous self-experience with previous labels. For example, a user that has previously felt “10 out of 10” high after taking a dose labelled “8” and that wished to stay below a “5 out of 10” high using a product labelled “8” would take a half dose.

$$proportion of dose = \frac{\begin{matrix}expected high \times \\ label on previously taken full dose\end{matrix}}{\begin{matrix}current dose label \times self - \\ assessment of previously taken dose\end{matrix}}$$

$$\frac{1}{2} = \frac{5 \times 8}{8 \times 10}$$

Persons of ordinary skill will readily appreciate that consistent use of the method across a series of products would practically require more than a single person involved in the benchmarking operation, i.e. a statistically significant sample of persons would likely be exposed to a full dose and the label provided to the drug would be a statistical derivation of the test results obtained from the sample, perhaps a mean or median.

### Alcohol Variant

EEG data was recorded from a female subject, aged 27, who self-reported her level of alcohol impairment as 7 out of 10 at the time of recording. The recording was segmented into 10 second snippets which were passed through a band pass filter and a 60 Hz notch filter and snippets that contained noise or otherwise appeared unreliable were removed.

Thereafter, a class was allocated to each snippet by each algorithm, only one [Y] of the classes [Y/N] being associated with use of the drug, the results thereof being shown in Table 7

TABLE 7

| | Snippet | | | | | | | | | | | | | | | | | | | | | | | | | |
|---|---|---|---|---|---|---|---|---|---|---|---|---|---|---|---|---|---|---|---|---|---|---|---|---|---|---|
| Algorithm | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 |
| 1 | x | x | x | x | x | x | x | | x | x | x | x | x | x | x | x | x | | x | | x | x | x | x | x | x |
| 2 | x | x | x | x | x | x | x | x | x | | x | x | x | x | x | x | x | x | x | | x | x | x | x | x | x |
| 3 | x | x | x | x | x | x | x | x | x | x | x | x | x | x | x | x | x | | x | | x | x | x | x | x | x |
| 4 | x | x | x | x | x | x | x | | x | | x | | x | | | x | x | x | | | x | x | x | x | x | x |
| 5 | x | x | x | | x | x | x | x | x | x | x | x | x | x | x | x | x | | x | | x | x | x | x | x | x |
| Total | 5 | 5 | 5 | 4 | 5 | 5 | 5 | 3 | 5 | 3 | 5 | 4 | 5 | 4 | 4 | 5 | 5 | 2 | 4 | | 5 | 5 | 5 | 5 | 5 | 5 |

Thereafter, in respect of each snippet of the plurality of snippets, a finding of psychoactive effect was made if the one class is allocated to said each snippet in a proportion of the total classifications of the snippet that meets or exceeds a predetermined threshold of 40% [2], as shown in Table 8

TABLE 8

| | Snippet | | | | | | | | | | | | | | | | | | | | | | | | | |
|---|---|---|---|---|---|---|---|---|---|---|---|---|---|---|---|---|---|---|---|---|---|---|---|---|---|---|
| Algorithm | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 |
| Total | 5 | 5 | 5 | 4 | 5 | 5 | 5 | 3 | 5 | 3 | 5 | 4 | 5 | 4 | 4 | 5 | 5 | 2 | 4 | | 5 | 5 | 5 | 5 | 5 | 5 |
| Finding | y | y | y | y | y | y | y | y | y | y | y | y | y | y | y | y | y | y | y | | y | y | y | y | y | y |

Finally, the psychoactive effect of the alcohol was calculated at 95%, based upon the proportion of EEG snippets found to have psychoactive effect [25 “Y” out of 26]

### Further Variations

Whereas specific embodiments are herein shown and described, it will be evident that variations are possible, including but not limited to:
proportional classifiers could be used in the context of snippets labelled to a scale
EEG readings for the calibration subjects can be taken at a different self-assessed levels
EEG readings could have been labelled without reference to self-assessment and purely based upon, for example, driving skill, or performance of any other standardized task.
Different threshold levels can be used and can snippets of differing lengths algorithms other than those based upon machine learning could be used the number of algorithms used is subject to variance and it will be understood that a plurality of algorithms could be construed to be a single algorithms; it is also of course possible that a single algorithm could be identified
whereas specific feature sets are described, other features sets can be used
the method could be extended for use with other drugs such as those for pain, gas, cough, sinus and itch relief or other drugs commonly used recreationally, such as cocaine, heroin, amphetamines, etc.
whereas in the method described, data is labelled based on self-reported ‘intoxication level’, other more specific drug effects such as ‘euphoria’ or ‘relaxation’ or any other readily identifiable mental or emotional state could be labelled in a similar manner
whereas specific filtering techniques are described, many other well known and conventional filters could be used; and
other EEG devices, sampling protocols and probe placements could be used

Further, whereas the labelling method suggests a single reference, i.e. an individual or group, it will be apparent that multiple reference scales could be provided, i.e. the package could provide ratings for each of a plurality of statistically-similar groups of persons, which could be relied upon by a purchaser based upon similarity to the group.

Yet further, whereas in the description, references is made to EEG data capture “during performance” or contemporaneous with a task or the like, it will be appreciated that EEG data capture could be carried out in close temporal conjunction such that the level of impairment during the capture is reasonably likely to be similar to that experienced during the task or the like. Persons of ordinary skill will readily be able to ascertain periods of temporal disconnect that are unlikely to result in significant variance.

### Enhancements and Extensions

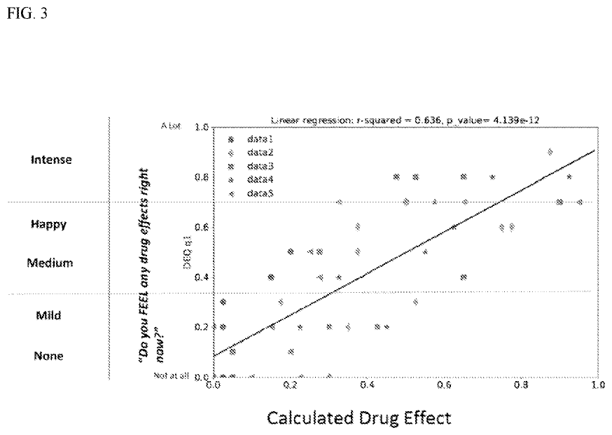

The drug effect magnitude calculation described above has been employed in an experiment wherein participants provide verbal reports of drug effect based upon the DEQ questionnaire (https://www.ncbi.nlm.nih.gov/pmc/articles/PMC3624068) wherein, in addition to providing a responses based on the DEQ participants also responded to the question “Do you feel any drug effects right now”, participants were encouraged to answer based upon the answers: none; mild; medium; and intense. The results of 5 participants are shown in FIG. 3. A regression calculation was carried out and a high degree of correspondence between the scales [p<0.00001] was demonstrated. As such, it is reasonable to predict that the method can be applied to permit users to reliably attain a drug effect according to the aforementioned scale, i.e. “none”, “mild”, “medium” and “intense” rather than the aforementioned somewhat arbitrary “8 out of 10” high mentioned above in paragraph [0039].

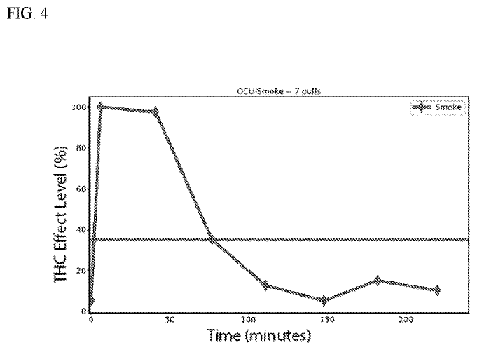

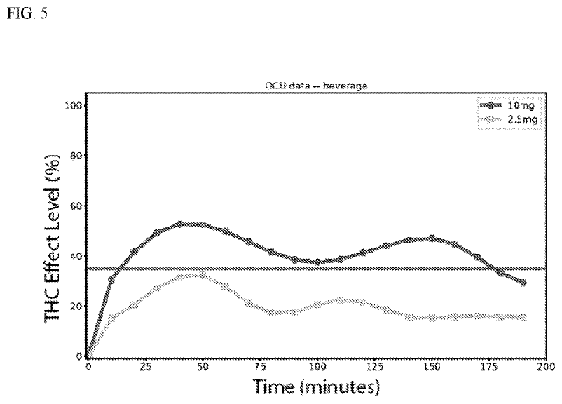

The drug effect magnitude calculation has also been employed in experiments wherein drug effect was measured at various times after consuming cannabis. FIG. 4 shows the results of a single participant smoking 7 puffs from a pipe, measured at t=o (before smoking) and t=6,41,77,148,182 and 220 minutes post intake. FIG. 5 shows the mean results of 6 participants after various times after intake of a THC beverage containing either 2.5 mg or 10 mg of THC.

The results demonstrate that the calculated drug effect follows a pattern well-established in the literature, namely, that smoking creates a brief, relative intense impairment that arrives and then dissipates quickly, whereas ingestion creates impairment that is longer to arrive, longer in duration, and lesser in intensity. As such, it is reasonable to predict that the method can be applied to permit users to reliably predict the timing of drug effects according to the aforementioned scale, i.e. none; mild; medium; and intense.

A method has also been developed to accommodate sampling data that varies in the interval between successive readings, to allow for timing variances that inevitably follow when headsets are used only intermittently (for comfort) during prolonged test periods. The method involves resampling the data in order to align it to the same time interval for all participants. To achieve that, a cubic spline interpolation can be applied to the original time series of THC level for each participant and the interpolated THC level at every 10 minute time interval can be averaged for participants of each user group. Assume the dataset has n+1 data points [(t0, y0), (t1, y1), . . . , (tᵢ, yᵢ), . . . (tₙyₙ)], where tᵢ is the time and yᵢ is the THC level of data point i (i=0, 1, . . . , n). Cubic spline interpolation fits the data points by a piecewise polynomial function S(t):

S(t)={C₁(t)x₀<x<x₁. . . Cᵢ(t)xᵢ₋₁<x<xᵢ. . . Cₙ(t)xₙ₋₁<x<xₙ

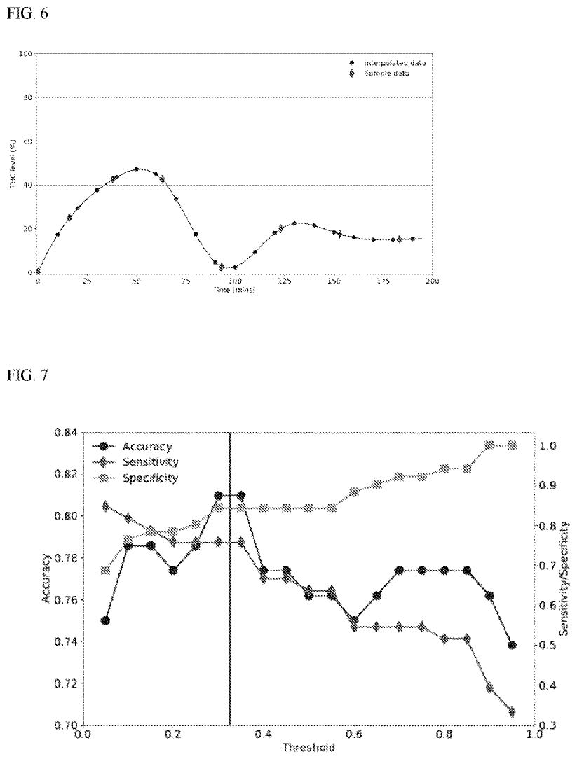

Cᵢ(t)=aᵢt³+bᵢt²+cᵢt+dᵢ is a cubic polynomial function with four parameters aᵢ, bᵢ, cᵢ, dᵢ which can be calculated analytically. Since the spline method fits to a piece of data rather than fit all data points at once, it avoids overfitting. FIG. 6 shows an example of the data resampling with cubic spline interpolation. 10 minutes was used as the time interval for the interpolated data to reduce the deviation from the original data; the deviation can be further smoothed by averaging the THC level for many participants.

Further, using an unbiased test dataset, containing 200 sessions labelled as either ‘intoxicated’ or ‘not intoxicated’ collected using the same methods described for collecting the training data set, except this data was held aside for testing and was not used to ‘train’ the algorithm, the method was used to generate a set of determinations of drug effect magnitude on a 0.0-1.0 scale, and utilizing a threshold, each of these determinations is classified as ‘intoxicated’ or ‘not intoxicated’ depending upon whether the magnitude of the drug effect is above or below the given threshold.

FIG. 7 shows accuracy, sensitivity and specificity calculated by utilizing various thresholds in the range 0.0-1.0 and calculating specificity, sensitivity and accuracy for each threshold according to the following formulas:
FP is the number of false positives found in the dataset, where a false positive is defined as classification of ‘intoxicated’ when it is the case that the participant was not intoxicated
TN is the number of true negatives found in the dataset.
TP is the number of true positives
FN is the number of false negatives
Total_Population is the number of elements in the dataset, in this case 200.

specificity=TN/(FP+TN)

sensitivity=TP/(TP+FN)

accuracy=(TP+TN)/Total_Population

The results surprisingly show that accuracy is maximized for a threshold of about 0.35, which is about the same point at which participants report a transition from “none” or “mild” drug effects to “moderate” drug effects. As such, it is reasonable to predict that the method can be reliably used to accurately discern moderate drug impairment.

In view of the foregoing, it should be appreciated that the invention should be understood to be limited only by the accompanying claims, purposively construed.

1. Method for defining the effect of a psychoactive drug to desired levels of sensitivity and accuracy, the method comprising the steps of:
using one or more algorithms to, in respect of each of a plurality of EEG snippets from a subject, allocate to each snippet a class selected from one or more class, each class being associated with a level of drug impairment,
the one or more algorithms each being defined by a model and a set of parameters and having been previously trained with a plurality of labelled EEG snippets, the labelled snippets including, for each of the classes, at least one snippet associated with a test subject experiencing the level of drug impairment associated with said each class,
creating a statistical model which provides the desired levels of sensitivity and accuracy based upon the proportion of snippets from test subjects having the known level of drug effect that falls within the associated class; and
defining the effect of the drug upon the subject by applying the statistical model to the classes allocated to the snippets of the subject.

2. A method according to claim 1, wherein the snippet length is about 5000 milliseconds.

3. A method according to claim 1, wherein the one or more algorithms is a plurality of unique algorithms.

4. A method according to claim 1, wherein the one or more algorithms is a plurality of machine learning algorithms.

5. Method for use with a psychoactive drug, the method comprising the steps of:
using a plurality of unique algorithms to, in respect of each of a plurality of EEG snippets, allocate to each snippet a class selected from a pair of classes, only one of the pair of classes being associated with use of the drug, each snippet being of a predetermined duration, the algorithms each being defined by a model and a set of parameters and having been previously trained with a plurality of labelled EEG snippets of the same predetermined duration, the labelled snippets including at least one snippet associated with a subject experiencing the psychoactive effect of the drug and labelled as the one of the pair of classes and at least one snippet associated with the subject when under no influence of the drug and labelled as the other of the pair of classes;
in respect of each snippet of the plurality of EEG snippets, making a finding of psychoactive effect if the one class is allocated to said each snippet in a proportion of the total classifications of the snippet that meets or exceeds a predetermined threshold; and
calculating the psychoactive effect of the drug based upon the proportion of EEG snippets found to have psychoactive effect.

6. Method according to claim 5, wherein the plurality of labelled EEG snippets are obtained by segmenting a plurality of EEG recordings.

7. Method according to claim 6, wherein the plurality of labelled EEG snippets are that which remain after an EEG recording has been segmented and segments determined to be unreliable or noisy through conventional filtering techniques have been removed.

8. Method according to claim 6, wherein the plurality of algorithms includes algorithms based upon each of a plurality of models.

9. Method according to claim 8, wherein, in respect of each model of the plurality, a plurality of algorithms are defined, each of such plurality having a unique set of parameters.

10. Method according to claim 5, wherein the algorithms are machine learning algorithms.

11. Method according to claim 5, wherein
the labelled snippets include snippets associated with a plurality of subjects; and
the labelled snippets include, in respect of each of the subjects, a plurality of snippets associated with the subject when the subject is experiencing the psychoactive effect of the drug and a plurality of snippets associated with the subject when under no influence of the drug.

12. Apparatus comprising:
apparatus for collecting EEG data from a test subject and segmenting same into segmented EEG test data; and
a computing facility adapted to carry out the method of claim 5, wherein the plurality of EEG snippets used in the method are defined by the segmented EEG test data.

13. Apparatus comprising:
apparatus for collecting EEG data from a test subject, segmenting same into segmented EEG test data and removing therefrom, using conventional filtering techniques, segments determined to be one or more of unreliable and noisy, thereby producing filtered segments; and
a computing facility adapted to carry out the method of claim 5, wherein the plurality of EEG snippets used in the method are defined by the filtered segments.

14. Method for use in a workplace, the method comprising the steps of:
in respect of a plurality of performances of a task, each task being permed by a person, in circumstances such that the task is performed by persons under varying levels of drug influence, capturing EEG data of the persons during the performance of the task;
choosing one of the performances as the minimum level of competence permissible in the workplace based upon an assessment of the performance for competency; and
using the method of claim 5 to determine the extent to which the performer of the chosen task was under the influence of the drug and the permissible extent of drug influence in the workplace for persons responsible for carrying out the task.

15. Method for labelling a dosage of a psychoactive drug, the method comprising the step of:
administering a dose of the drug to a person;
collecting EEG data from the person, at one or more time intervals after intake;
using the method of claim 5 to calculate the maximum psychoactive effect the person experienced following the dose, the time of onset of the dose, where this time is the time required for the psychoactive effect to reach a threshold and the duration of the dose, where this time is the time between when the dose is administered and when the psychoactive effect recedes below the threshold and
labelling the dose with the calculated maximum, onset time, and duration.

16. Method according to claim 15, wherein the threshold is 0.35.
